(19) United States
(12) Patent Application Publication
(10) Pub. No.: US 2003/0152533 A1
(43) Pub. Date: Aug. 14, 2003

(54) NOVEL INGREDIENT AND USE OF SAME

(75) Inventor: Diana Tang, Suffern, NY (US)

Correspondence Address: Rupa Sen, Avon Products Inc, Avon Place, Suffern, NY, 10901, US

(73) Assignee: AVON PRODUCTS, INC., NEW YORK, NY

(21) Appl. No.: 10/032,835

(22) Filed: Dec. 27, 2001

(51) Int. Cl. A61K 7/04; A61K 7/025; A61K 7/06; A61K 7/11

(52) U.S. Cl. 424/61; 424/64; 424/70.17

(57) ABSTRACT

There is provided a method and composition for improving the water resistance, shine, gloss, surface smoothening, and/or film-forming attributes in various personal care, hair care and cosmetic products. The composition has a novel polyurethane polyesterpolyquat. Preferably, the polyurethane polyesterpolyquat is incorporated into a shampoo, hair conditioner, mascara, color cosmetic, body wash, liquid soap, hair color, nail care and/or skin care composition.

### BACKGROUND OF THE INVENTION

1. Field of the Invention

The present invention relates to a novel ingredient and use of same. More particularly, it relates to a novel polymer having a multiple number of urethane moieties, ester linkages, amides, and quaternary functionalities (hereinafter “polyurethane polyesterquat”). Even more particularly, the present invention relates to the use of polyurethane polyesterpolyquat in a variety of personal care, hair care and cosmetic products to provide water resistance, shine, gloss, surface smoothening, and/or film-forming benefits.

2. Description of the Prior Art

Water resistance, shine, gloss, surface smoothening, and film-forming are all attributes that are desired in many personal care, hair care and cosmetic products, preparations and compositions. For example, water resistance is a much-desired quality amongst consumers of color cosmetics, lipsticks, eyelash mascara, insect repellents and sunscreen preparations. Additionally, shine and gloss are much desired attributes in nail color and hair care preparations. Surface smoothening may be important in a shampoo, hair conditioner, styling mousse and other hair treatment preparation. This attribute may provide for more superior wet and dry hair combing. Film-forming is a desired attribute in a body wash, liquid soap, hair color and skin care preparation, amongst other products.

There is a desire for a single ingredient that, when incorporated into such compositions, can deliver multi-functionality to the formulated composition, particularly the aforementioned desired attributes or benefits. This result is now attainable in a composition that simply has an effective amount of a single compound, namely a polyurethane polyesterpolyquat. This single compound delivers multiple performance advantages to a composition that heretofore required the use of a number of different ingredients. Furthermore, these performance advantages have not been achieved by known monomeric esterquats.

### SUMMARY OF THE INVENTION

It is an object of the present invention to provide compositions that have water resistance, shine, gloss, surface smoothening and/or film-forming properties.

It is another object of the present invention to provide a composition having a polyurethane polyesterpolyquat.

It is a further object of the present invention to provide a method of topically applying a composition containing a polyurethane polyesterpolyquat to a substrate, such as skin, hair, lips or nails, to provide water resistance, shine, gloss, surface smoothening and/or film-forming to such substrate.

These and other objects and advantages of the present invention are achieved by a composition having a polyurethane polyesterpolyquat. The polyurethane polyesterpolyquat composition is a personal care, hair care or cosmetic composition.

### DETAILED DESCRIPTION OF THE INVENTION

The present invention relates to the formulation of a new compound and its use as or in a personal care, hair care or cosmetic composition. The compound is a polyurethane polyesterpolyquat. The present polyurethane polyesterpolyquat combines together a multiple number of urethane moieties, ester linkages, amides and quaternary functionalities, namely quaternized groups. This unique structure allows the polyurethane polyesterpolyquat to provide multiple benefits all at once.

The preferred polyurethane polyesterpolyquat is 1-Amino-2-Trimethylnol Ricinoleate/IDPI Copolymer-Diethyl Sulfate. Its chemical structure is:

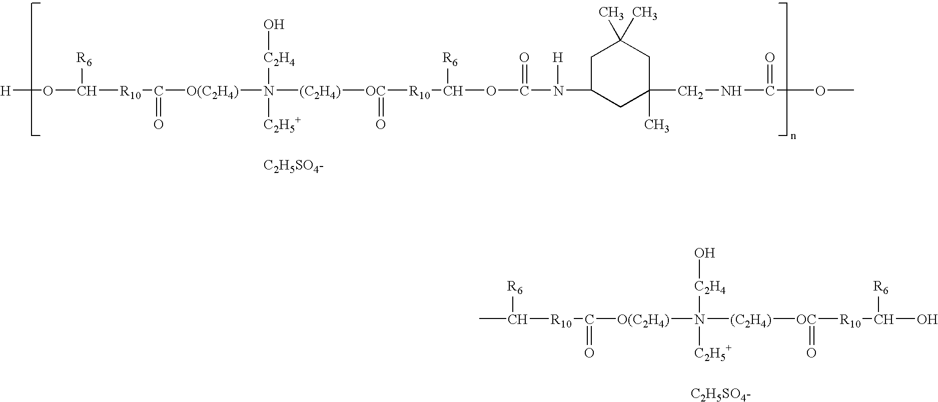

wherein R₆ is C₆H₁₃, R₁₀ is CH₂CH═CH(CH₂)₇, and n=4 to 40, preferably 8 to 25, more preferably 10 to 15.

The process of synthesizing the above polyurethane polyesterpolyquat is as follows. The first step is an esterification of TEA (triethanolamine) and ricinoleic acid to form esters called TEA Ricinoleate. The next step is the polymerization of TEA Ricinoleate with isophorone diisocyanate (IPDI) to form TEA diricinoleate/IPDI copolymer. The third step is the quaternization of the TEA diricinoleate/IPDI copolymer with the quaternizing agent diethyl sulfate to form the preferred polyurethane polyesterpolyquat.

A polyurethane polyesterpolyquat as described above, can be structurally generalized as follows:

H[(FA_{X}.TEA]-(IDPI-URETHANE)]ₙ-FA_{X}.TEA

wherein X=degree of esterification of TEA with Fatty Acid (FA) and ranges from 1 (monoester) to 3 (triester), but it is most preferably equal to 2 (i.e., the diester).

The chemical structures corresponding to the reaction steps of the preferred polyurethane polyesterpolyquat above are outlined below:

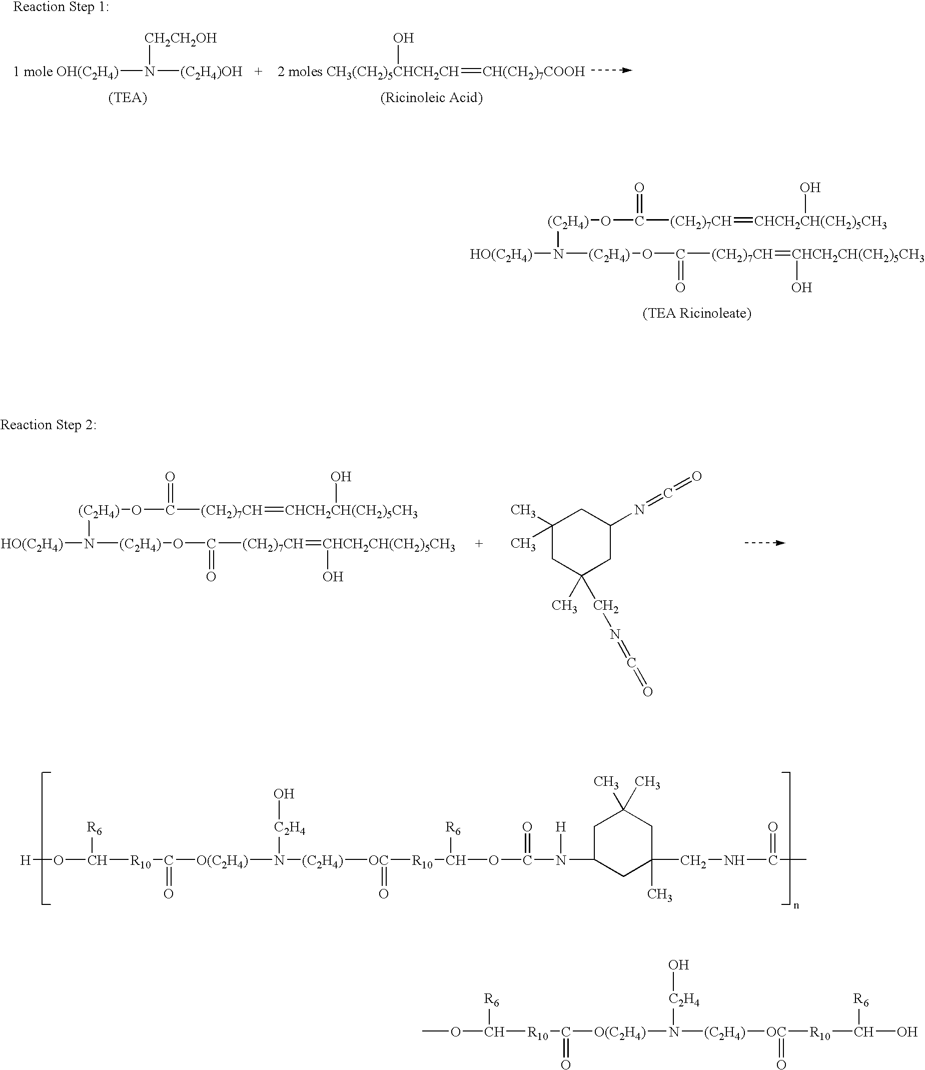

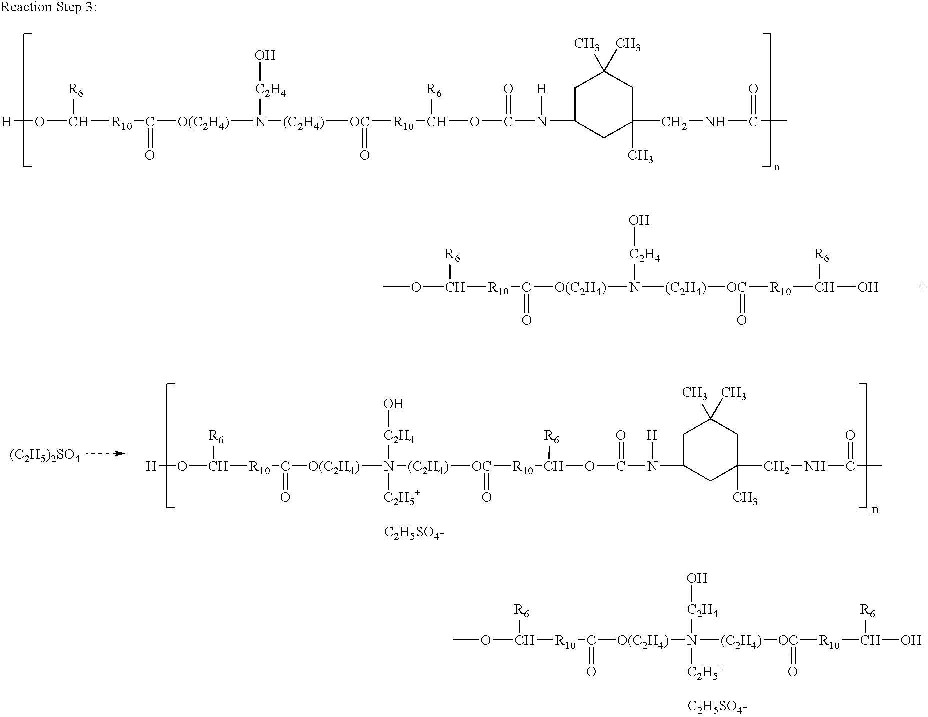

The chemical structure of this compound has several functional groups, such as ester bonds, amide bonds, carbamate bonds (urethane bonds) and hydrophobic modification, within the compound. Because of the multifunctional groups within the compound, this compound delivers multi-functionality and multiple benefits to the formulated products.

The polyurethane polyesterpolyquat of the present invention can be made with any fatty acid with free hydroxyl groups. Ricinoleic acid is preferred because it has a hydroxyl functional group that can be polymerized with IPDI. It is most preferred that a triethanolamine fatty acid diester is formed. The formation of the diester, rather than monoester or triester, can be facilitated by using two moles of fatty acid for every mole of triethanolamine. Even so, monoesters and triesters may form and thus be incorporated into the polyurethane polyesterpolyquat. Preferably, the polyurethane polyesterpolyquat includes over 50% diesters, more preferably over 75% diesters, and most preferably over 90% diesters. A high percentage of triester is less preferred, because the more triesters that the polyurethane polyesterpolyquat has incorporated therein, the more solid it will be.

The polyurethane polyesterpolyquat of the present invention may be added to personal care, hair care and cosmetic products, including, but not limited to, a shampoo, hair conditioner, hair styling mousse, mascara, color cosmetic, lipstick, body wash, liquid soap, hair color, nail care preparation (e.g. enamel), hair treatment preparation, and skin care preparation, in an amount effective to provide and/or enhance water resistance, shine, gloss, surface smoothening, and/or film-forming.

The compositions of the present invention preferably have the polyurethane polyesterpolyquat present in an amount about 0.005 weight percent or percentage by weight (wt %) to about 40 wt % based on the total weight of the composition. More preferably, the present compositions have polyurethane polyesterpolyquat present in an amount about 0.01 wt % to about 20 wt %. Most preferably, the present compositions have polyurethane polyesterpolyquat present in an amount about 0.1 wt % to about 10 wt % based on the total composition.

When used in a hair conditioner composition, the composition preferably has the polyurethane polyesterpolyquat present in an amount about 0.1 wt % to about 8 wt %, and more preferably in an amount about 0.5 wt % to about 3 wt %, based on the total weight of the composition. The polyurethane polyesterpolyquat provides for superior conditioning compared to conventional monomeric esterquats. Further, such hair care compositions that include the polyurethane polyesterpolyquat provide for longer-lasting styling hold, measured by curl retention, at high humidity conditions. Additionally, in hair care products, the instant polyurethane polyesterpolyguat helps deliver both conditioning and styling benefits, even from a single rinse-off product.

When used in a hair styling composition, the composition most preferably has a polyurethane polyesterpolyquat present in an amount about 0.3 wt % to about 10 wt %, based on the total weight of the total composition. Optimally, when the hair styling composition is a styling gel, the polyurethane polyesterpolyquat is present in an amount about 0.3 wt % to about 1 wt %, based on the total weight of the composition.

When used in a body wash composition, the composition more preferably has the polyurethane polyesterpolyquat present in an amount about 0.5 wt % to about 4 wt %, based on the total weight of the composition.

The compositions of the present invention may include one or more additional components, such as one or more antimicrobials, antioxidants, buffering agents, chelating agents, colorants, conditioning agents, emollients, emulsifiers (e.g., anionic, cationic or nonionic), film formers, fragrances, humectants, lubricants, moisturizers, pigments, preservatives, skin penetration enhancers, stabilizers, surfactants, thickeners, viscosity modifiers, vitamins, or any combinations thereof.

The hair care compositions of the present invention may be of the type including, but not limited to, a conditioning shampoo, a rinse-off conditioner, a leave-on conditioner, mousse, styling gel, hair color preparation or any combinations thereof.

When in the form of a shampoo, the hair care compositions of the present invention typically have one or more surfactants present in an amount required to clean the hair, preferably about 5 wt % to about 20 wt % based on the total weight of the composition. The surfactants in shampoo compositions may include anionic, nonionic or amphoteric surfactants, or mixtures of these surfactants. When in the form of a leave-on conditioner, the compositions of the present invention preferably have a relatively smaller amount of a non-ionic surfactant typically in the range about 0.01 wt % to about 3.0 wt % based on the total weight of the composition. When in the form of a leave-on conditioner, the compositions preferably have a fatty alcohol and an ethoxylated fatty alcohol having from about 12 to about 36 carbon atoms. Such fatty alcohol and ethoxylated alcohol are typically present in an amount about 0.01 wt % to about 5.0 wt % based on the total weight of the composition. Examples of such fatty alcohols include, but are not limited to lauryl alcohol, cetyl alcohol, stearyl alcohol, behenyl alcohol, or any combinations thereof. Examples of such ethoxylated fatty alcohols include all fatty alcohols with the ethoxylation from 2 moles to 30 moles.

The present compositions typically have a vehicle. The vehicle should be a physiologically acceptable or suitable vehicle. A “physiologically acceptable vehicle” or a “suitable vehicle” means any vehicle for a drug, cosmetic or medicament that is suitable for use in direct, safe contact with human tissues. The polyurethane polyesterpolyquat is preferably incorporated into a suitable topical vehicle to form a topical formulation prior to applying.

The compositions of the present invention may have a number of different product forms. Such suitable product forms include a cream, aerosol or pump spray, mousse, foam, gel, lotion, stick, pomade, solution, or incorporated into a patch or towelette.

The following are examples of compositions having the polyurethane polyesterpolyquat.

| | |
|---|---|
| Ingredient | Weight % |

Shampoo

| | |
|---|---|
| Distilled water | QS |
| Guar | 0.4 |
| Hydroxypropyltromonium | |
| Chloride | |
| citric acid | 0.1 |
| Disodium EDTA | 0.1 |
| Methylparaben | 0.2 |
| Cocamide MEA | 2.0 |
| Glycol Distearate | 0.75 |
| Na Lauryl Ether Sulfate | 18.0 |
| Polyurethane | 1.0 |
| polyesterpolyquat | |
| Fragrance | 0.5 |
| Methylisolhiazolinone, | 0.066 |
| Methylchloroisothiazolinone | |

Conditioner

| | |
|---|---|
| Distilled water | QS |
| HEC and Dialkyldimethyl | 0.25 |
| Ammonium Chloride | |
| Polyurethane | 2.0 |
| polyesterpolyquat | |
| citric acid | 0.3 |
| Disodium EDTA | 0.1 |
| Methylparaben | 0.2 |
| Cetearyl alcohol/Cetearth-20 | 0.3 |
| Stearamidopropyl | 0.5 |
| Dimethylamine | |
| Cetyl Alcohol | 2.0 |
| Cetyl/stearyl Alcohol | 4.0 |
| Propylparaben | 0.1 |
| Phenoxyethanol | 0.5 |
| Fragrance | 0.75 |

Mousse

| | |
|---|---|
| Distilled water | QS |
| Imidazolidinyl Urea | 0.5 |
| HEC and Dialkyldimethyl | 3.0 |
| Ammonium Chloride | |
| Polyurethane | 1.0 |
| polyesterpolyquat | |
| Isoceteth-20 | 0.5 |
| Fragrance | 0.3 |

Gel

| | |
|---|---|
| Distilled water | 91.6 |
| Disodium EDTA | 0.1 |
| Imidazolidinyl Urea | 0.5 |
| Carbomer | 0.5 |
| Aminomethyl Propanol | 0.5 |
| PVPVA copolymer | 6.0 |
| Polyurethane | 0.5 |
| polyesterpolyquat | |
| Fragrance | 0.3 |

Body Wash

| | |
|---|---|
| Polyurethane | 1.0 |
| polyesterpolyquat | |
| Fatty acids (e.g., Lauric | 20.5 |
| acid, Myristic acid, | |
| Palmitic acid, Stearic | |
| acid) | |
| Glycerin | 5.0 |
| Potassium hydroxide | 5.0 |
| Sodium chloride | 2.7 |
| Sodium auroamphoacetate | 2.5 |
| Propylene glycol | 1.5 |
| Glycol distearate | 1.25 |
| Fragrance | 1.0 |
| PEG-6 | 0.5 |
| Triclosan | 0.3 |
| Imidazolidinyl urea | 0.2 |
| Tetrasodium EDTA | 0.05 |
| Distilled water | QS |

Lipstick

| | |
|---|---|
| Polyurethane | 0.5 |
| Polyesterpolyquat | |
| Fatty acid esters (e.g., | 22.5 |
| Octyl palmitate, Isopropyl | |
| palmitate, Cetyl lactate, | |
| Caprylic/Capric | |
| triglyceride | |
| Wax (e.g., Beeswax, | 18.0 |
| Candellila, Ozokerite, | |
| Carnuba) | |
| Octyl dimethyl PABA | 3.0 |
| Lecithin | 0.3 |
| Allantoin | 0.2 |
| Preservatives (e.g., BHT, | 0.02 |
| BHA) | |
| Pigments (e.g., D&C Red | 11.0 |
| No. 6 Barium Lake, | |
| D&C Red | |
| No. 27 Aluminum Lake, D&C | |
| Red No. 21 Aluminum Lake, | |
| D&C Red No. 7 Calcium | |
| Lake) | |
| Titanium dioxide | 1.0 |
| Oil (e.g., Castor oil, | QS |
| Sesame oil, Soybean oil, | |
| Hydrogenated soybean oil, | |
| Lanolin oil) | |

Mascara

| | |
|---|---|
| Polyurethane | 0.3 |
| polyesterpolyquat | |
| Pentaerythrityl | 9.0 |
| tetrastearate | |
| Waxes (e.g., Beeswax, | 12.5 |
| Ozokerite, Candelilla, | |
| Carnuba) | |
| Glyceryl stearate | 4.7 |
| PVP/Eicosene | 4.25 |
| Oleic acid | 2.8 |
| Polyisobutane | 2.6 |
| Ammonium acrylates | 1.95 |
| copolymer | |
| Triethanolamine | 1.7 |
| Sorbitan sesquioleate | 1.0 |
| Benzyl alcohol | 0.8 |
| Vitamins (e.g., Panthenol, | 0.5 |
| Tocopheryl acetate calcium | |
| pantothenate) | |
| Sodium silicoaluminate | 0.5 |
| Sodium hexametaphosphate | 0.4 |
| Preservatives (e.g., | 0.6 |
| Methylparaben, | |
| Propylparaben) | |
| Propylene glycol | 0.25 |
| AMP-Isostearic hydrolyzed | 0.1 |
| keratin | |
| Sodium lauryl sulfate | 0.05 |
| SD alcohol 40 | 0.02 |
| Potassium sorbate | 0.02 |
| Propylene glycol stearate | 0.01 |
| EDTA | 0.005 |
| Pigments (e.g., | 14 |
| Ultramarines, Carmine) | |
| Distilled water | QS |

It should be understood that the foregoing description is only illustrative of the present invention. Various alternatives and modifications can be devised by those skilled in the art without departing from the present invention. Accordingly, the present invention is intended to embrace all such alternatives, modifications and variations that fall within the scope of the appended claims.

What is claimed is:

1. A composition comprising a polyurethane polyester polyquat compound and a vehicle suitable for topical application to hair, lips, skin and/or nails.

2. The composition of claim 1, wherein the polyurethane polyester polyquat is present in an amount effective to provide at least one benefit selected from the group consisting of water resistance, shine, gloss, surface smoothening, and film-forming.

3. The composition of claim 1, wherein said composition is in a form selected from the group consisting of a cream, a foam, a gel, a lotion, a solution, a pomade, a mousse, a stick, a pump spray, an aerosol spray, and any combinations thereof.

4. The composition of claim 1, wherein said composition is incorporated into a form selected from the group consisting of a patch, a towelette, and any combinations thereof.

5. The composition of claim 1, wherein the polyurethane polyester polyquat is present in an amount about 0.005 wt % to about 40 wt % based on the total weight of the composition.

6. The composition of claim 1, wherein the polyurethane polyester polyquat is present in an amount about 0.01 wt % to about 20 wt % based on the total weight of the composition.

7. The composition of claim 1, wherein the polyurethane polyester polyquat is present in an amount about 0.1 wt % to about 10 wt % based on the total weight of the composition.

8. The composition of claim 1, further comprising one or more additional components selected from the group consisting of antimicrobials, antioxidants, buffering agents, chelating agents, colorants, conditioning agents, emollients, emulsifiers, film formers, fragrances, humectants, lubricants, moisturizers, pigments, preservatives, skin penetration enhancers, stabilizers, surfactants, thickeners, viscosity modifiers, vitamins, and any combinations thereof.

9. The composition of claim 1, wherein the composition is selected from the group consisting of hair conditioner, shampoo, styling mousse, hair treatment preparation, lipstick, mascara, other color cosmetic, body wash, liquid soap, hair color, nail care preparation, skin care preparation, and any combinations thereof.

10. The composition of claim 9, wherein the composition is a hair conditioner, and wherein the polyurethane polyester polyquat is present in an amount about 0.1 wt % to about 8 wt % based on the total weight of the composition.

11. The composition of claim 10, wherein the polyurethane polyester polyquat is present in an amount about 0.5 wt % to about 3 wt % based on the total weight of the composition.

12. The composition of claim 9, wherein the composition is a hair styling product, and wherein the polyurethane polyester polyquat is present in an amount about 0.3 wt % to about 10 wt % based on the total weight of the composition.

13. The composition of claim 9, wherein the composition is a styling gel, and wherein the polyurethane polyester polyquat is present in an amount about 0.3 wt % to about 1.0 wt % based on the total weight of the composition.

14. The composition of claim 9, wherein the composition is a body wash, and wherein the polyurethane polyester polyquat is present in an amount about 0.5 wt % to about 4 wt % based on the total weight of the composition.

15. A method of imparting to a substrate selected from the group consisting of hair, skin, lips and nails an attribute selected from the group consisting of water-resistance, shine, gloss, surface smoothness, film formation, and any combinations thereof, the method comprising applying to the substrate an effective amount of the composition of claim 1.

16. The method of claim 15, wherein the composition is selected from the group consisting of hair conditioner, shampoo, styling mousse, hair treatment preparation, lipstick, mascara, color cosmetic, body wash, liquid soap, hair color, nail care preparation, skin care preparation, and any combinations thereof.

17. The method of claim 15, wherein the composition is selected from the group consisting of lipstick, mascara, and color cosmetic, and wherein the attribute is water-resistance.

18. The method of claim 15, wherein the composition is selected from the group consisting of hair conditioner, shampoo, styling mousse, hair treatment preparation, hair color, nail care preparation, and any combinations thereof, and wherein the attribute is selected from the group consisting of shine, gloss, and any combination thereof.

19. A method of enhancing the appearance of hair comprising applying to the hair an effective amount of the composition of claim 1.

20. A method of styling hair comprising applying to the hair an effective amount of the composition of claim 1.

21. The method of claim 20, wherein said hair has improved styling hold.

22. The method of claim 20, wherein the hair has improved curl retention.

23. The method of claim 20, wherein the polyurethane polyester polyquat is present in an amount about 0.3 wt % to about 10 wt % based on the total weight of the composition.

24. A method of conditioning hair comprising applying to the hair an effective amount of the composition of claim 1.

25. The method of claim 24, wherein the hair has improved smoothness.

26. The method of claim 24, wherein the polyurethane polyester polyquat is present in an amount about 0.1 wt % to about 8 wt % based on the total weight of the composition.

27. A method of cleansing skin comprising applying to the skin an effective amount of the composition of claim 1.

28. The method of claim 27, wherein the polyurethane polyester polyquat is present in an amount about 0.5 wt % to about 4 wt % based on the total weight of the composition.

29. A compound having the formula: 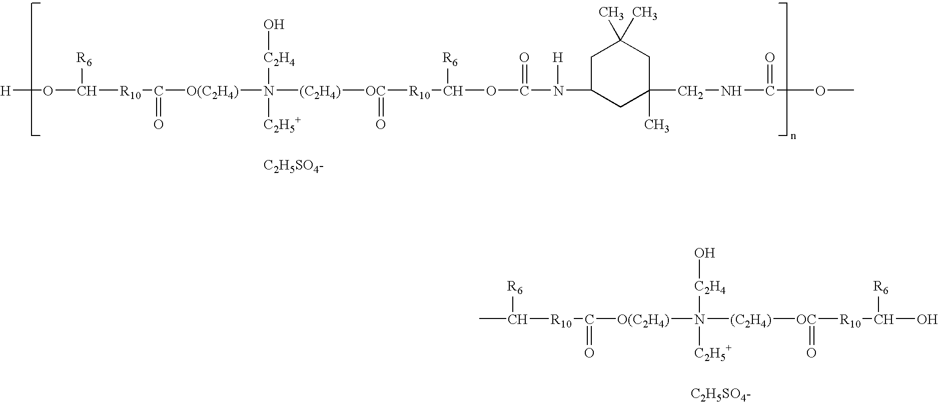
wherein R₆ is C₆H₁₃, R₁₀ is CH₂CH═CH(CH₂)₇, and n=4 to 40.

30. The compound of claim 29, wherein n=8 to 25.
